(19) United States
(12) Patent Application Publication
(10) Pub. No.: US 2011/0061345 A1
(43) Pub. Date: Mar. 17, 2011

(54) SOLID AND SEMI-SOLID DOSAGE FORMS AND SYSTEMS AND METHODS FOR FORMING AND PACKAGING THEREOF

(76) Inventors: S. Rao CHERUKURI, Vienna, VA (US); Mayur PHADKE, Sterling, VA (US); Revanth Babu MUTYALA, Rockville, MD (US); Shafi SHAIK, Frederick, MD (US)

(21) Appl. No.: 12/826,200

(22) Filed: Jun. 29, 2010

Related U.S. Application Data
(60) Provisional application No. 61/221,386, filed on Jun. 29, 2009.

(51) Int. Cl. B65B 1/04; A61K 9/16; A61P 29/00; A61P 23/00

(52) U.S. Cl. 53/473; 424/489

(57) ABSTRACT

The present invention relates to a pharmaceutical dosage form, and methods and system for making the pharmaceutical dosage form. The pharmaceutical dosage form contains a first component and a second component sealed in a container, such as a packet or sachet, blister pack, and capsule. Machines and methods for making the pharmaceutical dosage form is also provided.

This application claims the priority of U.S. Provisional Patent Application No. 61/221,386, filed Jun. 29, 2009, which is incorporated herein by reference.

### FIELD OF THE INVENTION

The present invention relates to a solid/semi solid dosage form packaging, and methods and system for making the solid/semi solid dosage form packaging.

### BACKGROUND OF THE INVENTION

Pharmaceutical compositions may be produced in a variety of dosage forms, depending upon the desired route of administration of the active ingredient. Oral dosage forms, for example, include such solid/semi-solid compositions as tablets, granules, powders, beads, minitablets, and pellets. The particular dosage form utilized will depend on such factors as the solubility and chemical reactivity of the active ingredient. Further, the dosage form may be selected so as to optimize delivery of the active ingredient and/or consumer acceptability of the composition.

Also, the accuracy of the doses is of importance. Pharmaceutical compositions often contain active drug components that are harmful if the given dose is too high. On the other hand, the desired effect of the medicament is not achieved if the dose is too low.

When two or more ingredients are contained in a dosage form, uniform mixing of the ingredients prior to packaging is important in maintaining consistency and accuracy of the doses. It is, however, difficult to uniformly mix two or more solid ingredients having different physical characteristics, such as particle size, density, and flowability, as the ingredients tend to separate.

Therefore, a need remains for providing consistent and accurate pharmaceutical dosages, especially for rapid melt dosages. Such compositions are ideal for uses in the fields of pediatric and geriatric care, that is, for use with people or mammals who has severe health issues, who can not swallow the tablet or capsule, and people who do not have any teeth. Such compositions are also used in the cancer patients. Such compositions can be administered without water.

### SUMMARY OF THE INVENTION

By individually metering and dispensing specific volumes of each component of the dosage, the amount of the components for each dose is secured with very high accuracy. Thus, one of the advantages with the present invention is that the accuracy and precision of the amount of the components can be monitored and metered, and the result used to secure the quality of the pharmaceutical product.

The present invention relates to a new method for dosing a pharmaceutical product in a sealed container, such as a packet, sachet and blister pack, comprising a first component and a second component. In an embodiment, the first component are rapid melt granules or bead, and the second component is an active ingredient. The rapid melt granules serve as carriers that allow the active ingredient to dissolve in the mouth and be swallowed without the administration of water. When in use, the user merely opens the container and empties its content into the mouth. The same principle can be used to make pharmaceutical products containing more than two components.

The two component dosage form can be packaged in a sealed container, such as a packet, sachet, blister pack, or other unit dose form. The method comprises providing the first component by weighing the volume of the first component. Thus, a defined dose of the first component is provided, for example beads. Further, the method comprises introducing the first component into a package, weighing the volume of the second component, introducing the second component into the package, and sealing the package. Subsequent components are also individually metered before being added to the package. When all the components are added to the container, it is sealed and ready for use.

The present invention also provides a machine, system, apparatuses, or devices for making the two or one component dosage form. The machine includes at least two hoppers, each for holding and dispensing a component of the final dosage form. Each hopper dispenses its content into a dosing wheel which includes holes on its peripheries. Thus, if there are two components in the final dosage form, each component is contained in a hopper which is associated with a dosage wheel. The wheel spins on its center axis and allows the component contained in the hopper to fill the holes. A scraper removes the excess component to ensure that the volume of the material is accurate. As the hole aligns with a funnel, its component empties into the funnel which funnels the ingredient into a package for forming the dosage form. The multiple wheels are timed so that the components are added to the funnel and packaged together. This way, the ingredients are individually metered and added to the packaged dosage form to ensure accuracy and consistency. Preferably, the amount of each component within the package varies no more than 5%, preferably no more than 2.5%, most preferably no more than 1.5% between batches and packages.

### BRIEF DESCRIPTION OF THE DRAWINGS

FIG. 1 is a picture showing an embodiment of the present invention.

FIG. 2 is a picture showing a dosing wheel.

FIG. 3 is a picture showing the bottom side of a dosing wheel with a closed cover.

FIG. 4 is a picture showing the bottom side of a dosing wheel with an opened cover.

FIG. 5 is a picture showing a dosing wheel with a scraper.

FIG. 6A is a drawing showing the top view of a dosing wheel.

FIG. 6B is a drawing showing a cross-section of a dosing cup.

FIG. 7 is a drawing showing the top view of a dosing wheel for handling two components.

### DETAILED DESCRIPTION OF THE PREFERRED EMBODIMENTS

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of such drugs, and reference to “an excipient” includes reference to one or more of such excipients.

As used herein, the terms “formulation” and “composition” and “component” are used interchangeably and refer to a mixture of two or more compounds, elements, or molecules. In some aspects the terms “formulation” and “composition” may be used to refer to a mixture of one or more active agents with a carrier or other excipients.

As used herein, “active agent,” “bioactive agent,” “pharmaceutically active agent,” “pharmaceutical,” “active ingredient” or “functional agents” variations thereof may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “blood level” may be used interchangeably with terms such as blood plasma concentration, plasma level, plasma concentration, serum level, serum concentration, serum blood level and serum blood concentration.

As used herein, “oral dosage form” and the like refers to a formulation that is ready for administration to a subject through the oral route of administration. Examples of known oral dosage forms, include without limitation, tablets, caplets, powders, pellets, granules, beads and mini tablets and combinations there of etc. Such formulations also include multilayered tablets wherein a given layer may represent a different drug. In some aspects, granules, powders, pellets, minitablet (under 7 mm in diameter, preferably under 5 mm, most preferably under 4 mm), or nanoparticles may be coated with a suitable polymer/fats/waxes/emulsifiers/carbohydrates or a conventional coating material to achieve, for example, greater stability in the oral cavity, gastrointestinal tract, to achieve the desired rate of release, or to improve taste. Tablets and caplets may be scored to facilitate division of dosing. Alternatively, the dosage forms of the present invention may be unit dosage forms wherein the dosage form is intended to deliver one therapeutic dose per administration. Particular embodiments or groups of embodiments may be expressly limited to subsets of these dosage forms.

As used herein, “packet” or “stick pack” refers to a small, sealed packet containing a quantity of material, which is a single-use or unit dose quantity.

As used herein, an “effective amount” or a “therapeutically effective amount” of a drug or active ingredient refers to a sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert and pharmaceutically acceptable material that has substantially no biological activity, and makes up a substantial part of the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

“Rapid melt granules,” as used herein, are granules, powders, beads, minitablets, pellets, nanoparticles, or combinations there off that, when placed in the mouth, dissolves within 60 second, preferably 30 seconds, more preferably 20 seconds, yet more preferably 10 seconds, and most preferably 5 seconds. The granules, powders, beads, minitablets, pellets, nanoparticles, or combinations thereof can contain an active ingredient, but that is not necessary. Rapid melt granules can be made using the process disclosed in U.S. Patent Application Publication No. 2010/0010101, which is incorporated herein by reference.

The dosage form of the present invention comprises at least a first and a second component that are provided in a sealed container. In an embodiment, the first component is a rapid melt granule and the second component is an active ingredient, preferably in powder, granule, beads, minitablets, pellets, nanoparticles, or combinations there off. The rapid melt granules acts as a carrier for the active ingredient so that, when the dosage form is placed in the mouth, the active ingredient can be rapidly dissolves and ingested without additional administration of water. Although water is not required to dissolve the active ingredient, in certain embodiment, water may be used to assist in ingestion of the dosage form.

The dosage from can be made by providing the first component by metering a desired volume of the first component. Thus, a defined dose of the first component is provided, for example beads. Further, the method comprises introducing the first component into a package, metering a desired volume of the second component, introducing the second component into the same package, sealing the package into which the components have been introduced. Subsequent components can similarly be added to the same package.

In another embodiment, the method comprises sealing the package after introducing the components.

In one embodiment, the present invention provides a solid or semi solid product comprising at least a first and a second component in the same dosage form. The product can be contained, e.g. in a package. Preferably, at least one of the components contains an active ingredient. It is also desirable to have least one of the components containing placebo rapid melt granules. The placebo rapid melt granules are preferably flavored. Preferably, the weight ratio of the active ingredient to the rapid melt granules is 0.1:100 to 100:0.1, preferably 1:2 to 1:7.

In another embodiment, the present invention provides a dosage form in a sealed container containing at least two components. The dosage form can be contained, e.g. in a package. Preferably, at least one of the components contains an active ingredient, while at least one of the other components contains placebo rapid melt granules, preferably flavor rapid melt granules.

The active ingredient delivery forms are preferably beads, mini tablets, granules, crystals, powder and combinations there off. The active ingredients can also be modified for taste masking, or controlled or sustained release. The active ingredient, as a component of the dosage form, can be uncoated or coated beads, mini tablets, granules, crystals or powder and combinations there off in the form of a unit dose packet. The taste masking of the active ingredients can be achieved with polymers (methacrylate polymers, cellulose polymers, or combinations there off), resins, fats, waxes, or carbohydrates. Processes for taste masking of the active ingredients include granulation, complexation, spray congealing, spray drying and fluid bed coating, which are known in the art.

In another embodiment, a machine is used for making the dosage form with least two components. The machine is preferably a volumetric feeder system that can be used to dose various pharmaceutical dosage forms. The machine contains at least two hoppers with corresponding dosing wheels for metering each component into a package. Preferably, the machine also includes mechanisms for forming and sealing the package, e.g. stick pack, as it is being filled. These mechanisms include, vertical forming jaws, horizontal sealing jaws, stick forming tube, and heaters.

The equipment can be used to accurately feed one, two or three different ingredients into a single unit dose. The dosing disc is made up of fixed or variable thickness to accommodate different fill weights. Volume of the dosing cup determines the fill weight of each ingredient into a packet (or stick). The accuracy of fill is achieved by measuring the density of the product being dosed and the required dosage. Volume of the cup is then determined to achieve the required fill weight. Based on the size of the granules/beads/powders/minitablets/pellets/nanoparticles and the thickness of the dosing disc, the diameter of the dosing hole is determined.

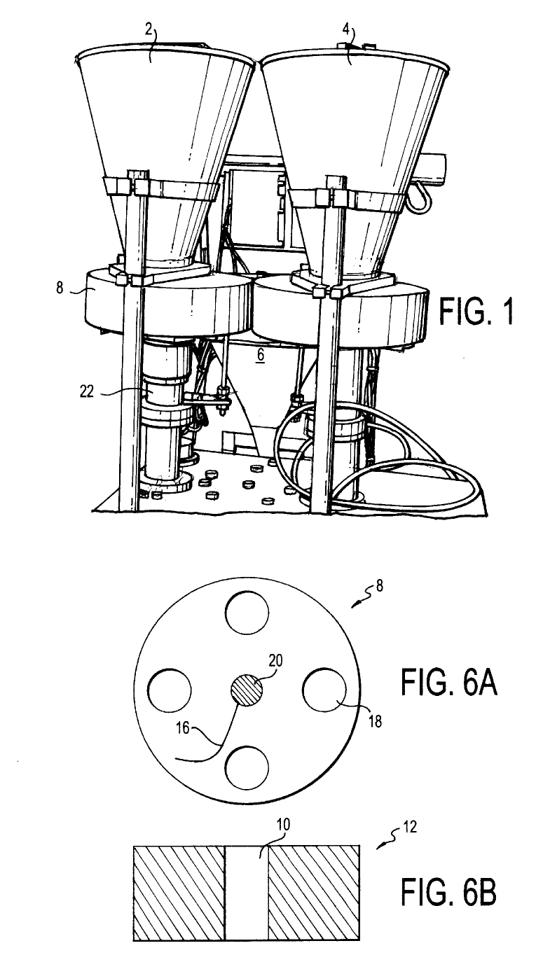

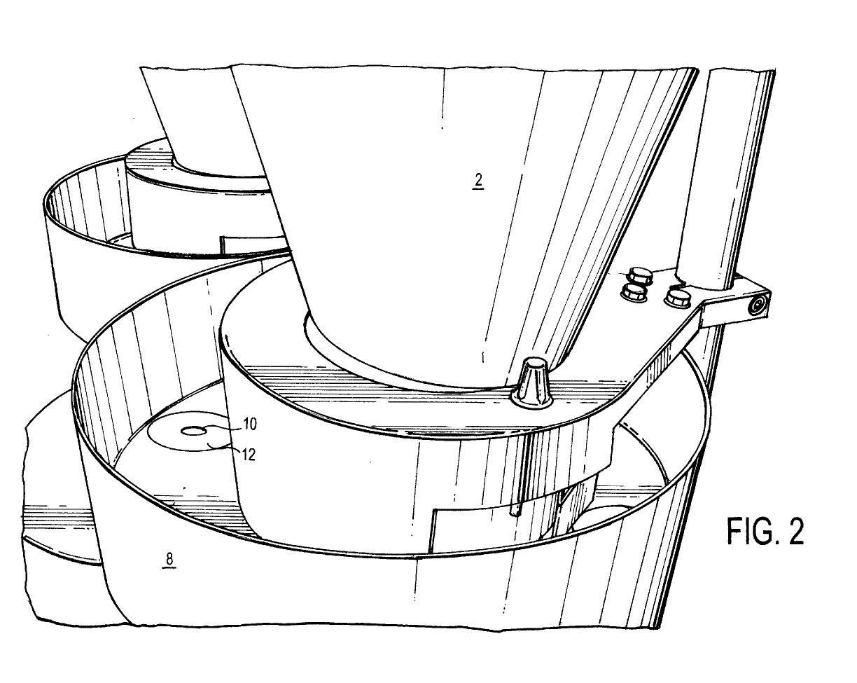

Referring to FIGS. 1-6 which show an embodiment of the machine for filling the dosage form, which includes hoppers 2 and 4. Although the Figures shows two hoppers for filling dosage forms containing two components, additional hoppers and associated components can be added to accommodate dosage forms having more than two components. The hoppers 2 and 4 contain different components of the final dosage form. For example, the first hopper 2 can contain a powdered active pharmaceutical ingredient, while the second hopper 4 can contain taste enhanced, e.g. flavor, rapid melt granules.

Each hopper 2 or 4 drains its component into a dosing wheel 8, preferably locating directly below the hopper. The dosing wheel 8 contains a plurality of dosing cups 12 in its periphery (FIG. 6A). The dosing cup (FIG. 6B) is essentially a cylindrical disc having a through hole 10 in its center for metering the component dispensed from the hopper 2 or 4. The hole 10 can be of different sizes depending on the desired amount of the component being dispensed into the final package. The dosing cups 12 fit into open recesses 18 in the dosing wheel 8, so that its can be removed/replaced to accommodate different amounts of the desired component. The volume of the component can be changed by changing the hole 10 diameter and/or the thickness of the dosing wheel 8. Because it is generally impractical to change the thickness of the dosing wheel 8, the volume of the component is determined by varying the diameter of the hole 10. Thus, to increase the amount of active ingredient, the operator removes the dosing cup 12 and replaces it with another dosing cup 12 having a hole 10 with a larger diameter. The desired volume of a component is calculated from its bulk density. Because each component is homogenous, its bulk volume is generally consistent and does not vary from batch to batch, as compared to heterogeneous mixtures of the components.

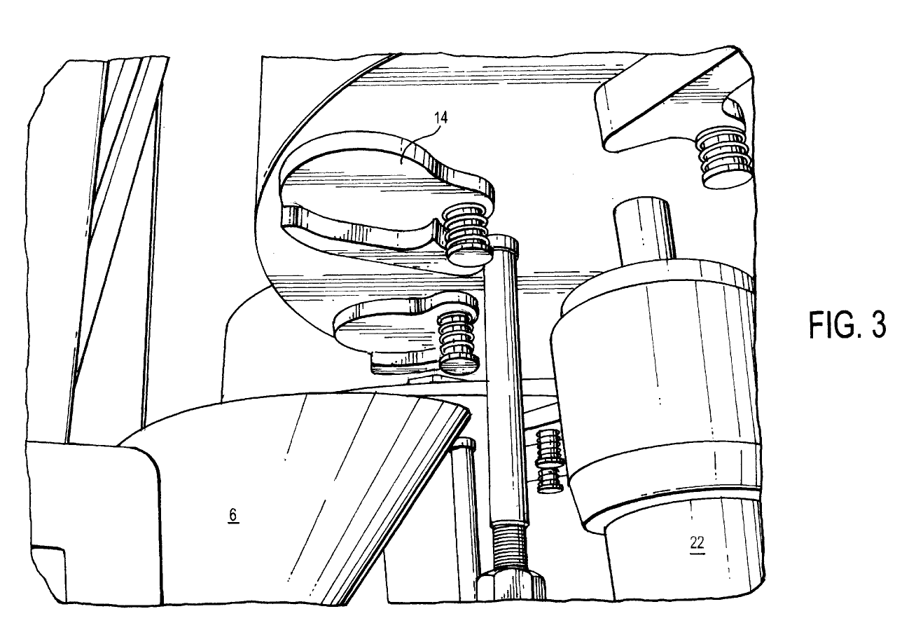

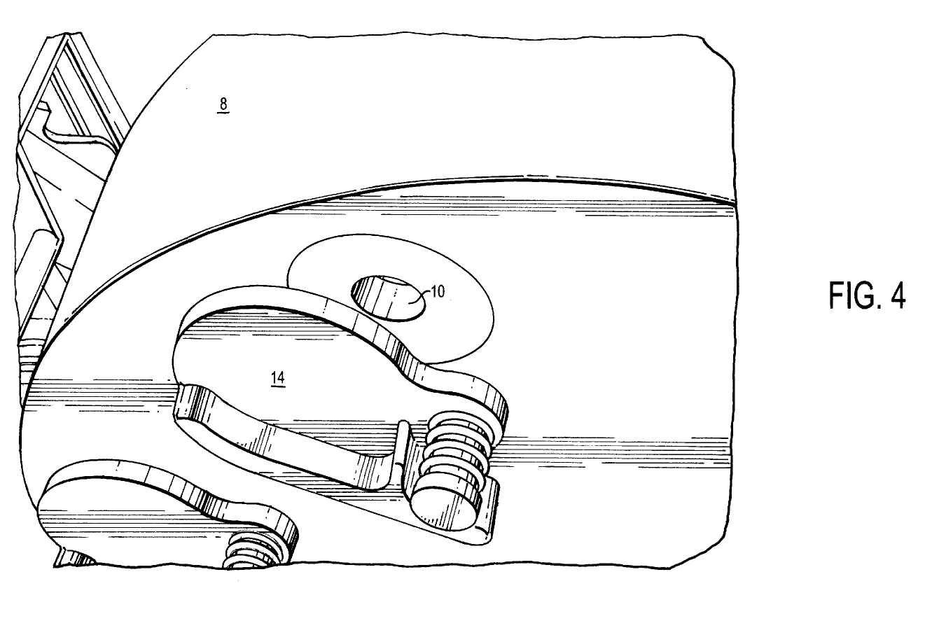

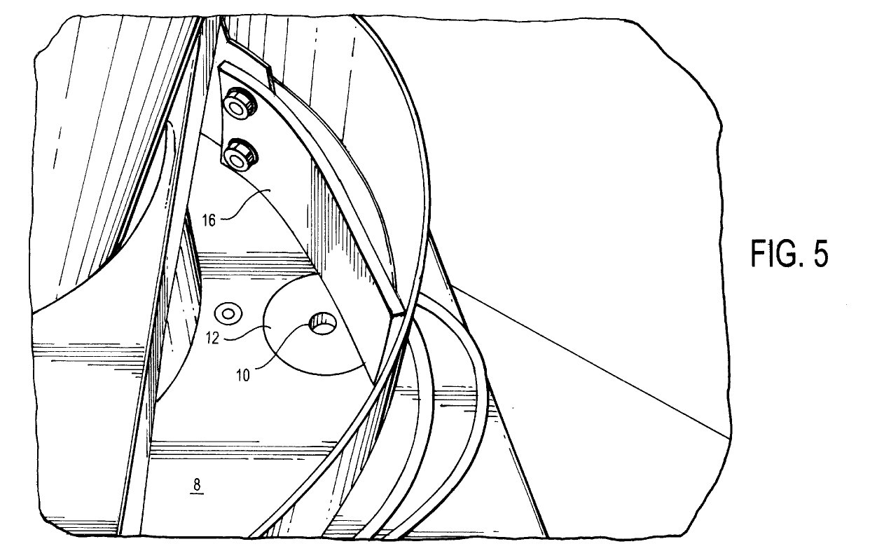

In operation, the dosing wheel 8 spins around its center axis 20 at a fixed speed while the desired component from the hopper 2 or 4 is being drained into the dosing wheel 8 to fill the holes 10. During each revolution, when a dosing cup 12 comes into alignment with a funnel 6, it empties its content into the funnel 6. Although “funnel 6” is used herein, it should be understood that the “funnel 6” needs not have the shape of a common funnel as long as it is capable of directing the components into their package. Prior to coming into alignment with the funnel 6, a scraper 16 (FIGS. 5 and 6A) scrapes excess material of the top of the dosing cup 12 such that the component is only contained within the volume of the hole 10, with no overfill. That way, a consistent and accurate amount of the component is ensured. Normally, the bottom of the hole 10 is closed by a cover 14 (FIG. 3). When the dosing cup 12 is in alignment with the funnel 6, the cover 14 opens (FIG. 4) revealing the hole 10 and, as a result, emptying the content of the dosing cup 12 into the funnel 6. The closing and opening of the cover 14 can be accomplished mechanically or by electronic control. Mechanical opening and closing can be accomplished, for example, by a pair of stationary posts. Just before the dosing cup 12 comes into alignment with the funnel 6, the door may be tripped into an open position with a first stationary post; and as it leaves the funnel 6, the cover 14 hits a second stationary post which pushes it into a closed position. Alternatively, electronic controls may be used to open and close the cover 14 when it is in alignment with the funnel 6. Mechanical opening and closing of the cover is generally preferred because it is a simpler system.

The power to spin the dosing wheel 8 is preferably provided via a drive shaft 22. Each dosing wheel 8 must be timed to synchronize with the other dosing wheel(s) so that the components are dispensed into the funnel 6 at substantially the same time. Preferably, this is accomplished by driving all dosing wheels 8 by a single motor. This way it is possible to spin the dosing wheels 8 at the same speed and to achieve proper delivery of the components to the funnel 6. In this preferred embodiment, it is desirable to include a clutch for each dosing wheel 8, so that it can be disengaged from the motor during operation for quality checks or repair.

Once the components are delivered to the funnel 6, they are directed to a container for packaging. The container can be, but not limited to, a package, a sachet, or a packet. Preferably, the container is a flexible package made from plastic, foil, glass, heat sealable laminated film (paper and polyethylene (PE), polyethylene terephthalate (PET) and PE, foil and PE, foil and paper and PE, PET and foil and PE), or combinations thereof. In certain embodiments, the machine also contains equipment for printing on the package, sealing (vertical and horizontal sealing jaws), heating, forming a tube from the packaging material, and cutting the package. Those equipments, when present, are synchronized with the dosing cups to properly package the final dosage form. In an example, the sealing jaws and cutter can be driven using the same motor driving the dosing wheel 8 to achieve synchronization. Alternatively, electronic controls can also be used to synchronize the equipment. Preferably, the final package has dimensions of 0.7 in. to 3.5 in. wide and 1 in. to 6 in. long.

In an embodiment, it is desirable to add an inert gas, such as nitrogen, to the package before sealing. This could be accomplished by blowing gas through the funnel 6 along with the components. This way, the gas is delivered through the funnel into the package before sealing. In an embodiment, the inert gas can be delivered to the funnel through a tube that connects a gas container to the funnel 6 inlet. Preferably, a valve is placed between the gas container and the funnel 6 to regulate the pressure of the gas entering the package. The regulated pressure is preferably between about 2 PSI to about 7.5 PSI, with minimum of about 50 PSI pressure in tank at all times

In an embodiment, to prevent clogging of the equipment by the components, vibrators can be placed, e.g., to provide vibration in the funnel 6, the dosing wheel 8, and/or the hoppers 2 and 4 to prevent clogging of the machine. Preferably, the vibration is 4,000 vibrations per minute (vpm) to 18,200 vpm. Other than vibration, the surfaces that come into contact with the components can also be coated to prevent clogging. For example, the surface can be coated with polytetrafluoroethylene (PTFE) to reduce friction and improve flow of the components. Other friction reducing techniques are also known in the art. Alternatively, the component is itself formulated to impart flowability, by adjusting the amount of excipients to accommodate formulation-related flowability. For example, a lubricant, e.g. alkaline stearates (preferably magnesium stearate), silicon dioxide, talc, or stearic acid, can be added to improve flow of a component.

In a preferred operation, the components are filled on to the dosing wheels 8 through hoppers 2 and 4. The dosing wheels 8 rotate at a set speed filling the components into the dosing cups 12 of their respective dosing wheels 8. Scrapers 16 removes overfill components on top of the cups and ensures accurate fill of the ingredient. The dosing wheels move further and empty their contents into a funnel 6 located below the wheel. The components are then dosed into a dosage form, preferably a flexible package. After filling of required ingredients the horizontal sealing jaws pull the stick downwards and seal and cut the stick from the top, while forming a second package and the process continues.

For example, a composition containing taste masked guaifenesin (beads, granules, powder, minitablets, pellets, nanoparticles, or combinations thereof) and flavored placebo rapid melt granules combination, two discs with two hoppers can be used. Speed of each disc is controlled using a single drive unit so that the contents of the cup are emptied into a single feed tube at the same time. Thus, all material is dosed into the stick to form a unit dose.

Although the drawings depict a two component dosage form, additional set-up can be made to dose three or more components into a final dosage form. Three hoppers, with three dosing discs will be used for a three component dose operation; four hoppers, with four dosing discs will be used for a three component dose operation; etc. Those dosage forms and machines for forming thereof are within the scope of the present invention.

In a preferred embodiment, the machine for used with the present invention has following specification:

| | |
|---|---|
| Pack style | Pillow type |
| Packing dimension | W2″ - minimum 15 mm to maximum 152 mm |
| | L2″ - minimum 60 mm to maximum 279 mm |
| Packing speed | 20-60 Bags/min |
| Max. film dimension | Ø12″ W 11″ |
| | Thickness 3 mil. |
| Power supply | 220 V, 60 Hz, 8 Kw |
| | single phase |
| Machine weight | 800 Lb |
| Machine dim. | W 20″ × L 30″ × H 92″ |

In another preferred embodiment, packaging machine has following features can also be used:

| Filling System | Adjustable telescopic Disc Volumetric Cup Filler | |
|---|---|---|
| Filling Accuracy | ±1 to 1.5% | |
| | Depend upon Nature of Product | |
| Type of Seal | 3 SideCentre or Back Seal | |
| Filling Range | 500 mg to 50 gm | |
| Packed Material | Granules and free flowing beads | |
| Size of Bag/Pouch | W - 100 to 160 mm | |
| | L - 80 to 130 mm | |
| Speed capacity | 40 to 70 bag/min. | |
| Machine size | H | 2150 mm |
| | W | 680 mm |
| | L | 900 mm |
| Wooden case | H | 1700 mm |
| | W | 1000 mm |
| | L | 1100 mm |

| | |
|---|---|
| Film roll size | O.D 250 to 300 mm max |
| | 1. D Film Reel Pipe |
| | 75 mm min. |
| Power consumption | Moto ½ H.P |
| | 220 V AC Single Phase |
| | 50 to 60 Hz |
| | Heater 150 × 2 + |
| | 300 × 2 |
| Film Quality | Laminated Roll + Aluminum Foil |

This packaging machine is specially designed to control weight variation of the components to +0.5% to +0.1%.

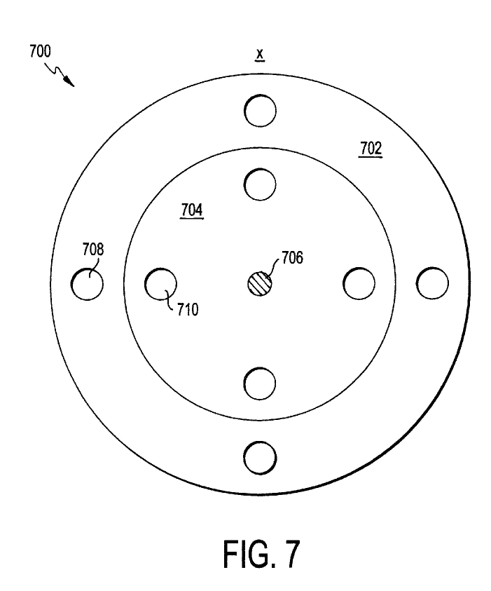

Although the above disclose two dosing wheels handling two components, it is possible to design one dosing wheel handling two components. Such a dosing wheel 700 is shown in FIG. 7, which contains two concentric circular portions. The outer portion 702 handles the first component, while the inner portion 704 handles the second component. The inner 702 and outer portions 704 are separated, e.g. by a wall, so that components in each portion do not intermix. The outer portion 702 contains recesses 708 for containing dosing cups, similar to the recesses 18 as previously described. The inner portion 704 also contains recesses 710 for containing dosing cups. Preferably, the recesses 708 and 710 are paired, such that each pair lays on a radius line of the dosing wheel 700. At the bottom of each recess 708, 710 is a cover, as previously disclosed for recesses 18, that can be opened to allow the content of the dosing cup to be emptied into a funnel. A stationary scrapers are preferably included to ensure accuracy and consistency as previously discussed. In this embodiment, a scraper is included in the inner portion 704 (inner scraper); and one is included in the outer portion 702 (outer scraper).

During operation, the dosing wheel 700 rotates on its axis by, e.g. a center shaft 706. A first hopper, containing the first component, drains its content into the outer portion 702; and a second hopper, containing the second component, drains its content into the inner portion 704. As the wheel rotates, just before the paired recesses 708 and 710 (and thus the corresponding dosing cups) reach a predetermined position where the recesses 708 and 710 are in alignment with the funnel, e.g. position x on FIG. 7, the scrapers remove overfill components on top of the dosing cups and ensure accurate fill of the component. When the recesses 708 and 710 reaches, e.g. position x. The covers below the recess pair open to allow the components contained in the dosing cups to empty into the funnel located below the dosing wheel. One the contents are emptied the covers are closed and the cycle continues. The mechanical or electronic control of the covers can be accomplished as previously disclosed. Once emptied into the funnel, the components can be processed a previously disclosed.

Although dosing wheel 700 has been disclosed above to handle two components, that wheel can be adapted to handle three or more components. In this embodiment, the wheel can be designed to include three or more concentric portions, where each component from a hopper is added to the a portion of the wheel.

The amount of active ingredient in the preparation is in the range of 0.1 mg to 10 g. Preferred prophylactic or therapeutic active ingredients contemplated for use in the present inventive subject matter are, without limitation, guaifenesin, mesalamine, diltiazem, metoprolol, balsalazide, aspirin, benzocaine, diphenhydramine, acetaminophen, ibuprophen and mixtures thereof.

Preferred prophylactic or therapeutic active ingredients contemplated for use in the present inventive subject matter are antibiotic, which is selected from the group consisting of amoxicillin and clavulanate potassium, ciprofloxacin HCl, azithromycin, clarithromycin, sterile ceftriaxone sodium, cefuroxime axetil, imipenem cilastatin, levofloxacin, ceftazidime, ampicillin sodium and sulbactum sodium, cefaclor, amoxicillin, cefdinir, roxithromycin, sterile cefotaxime sodium, vancomycin, piperacillin sodium and tazobactam sodium, morniflumate, flomoxef sodium, cefotiam dihydrochloride, ofloxacin, mupirocin calcium, vancomycin HCl, teicoplanin, cefadroxil monohydrate, sulbactum cefoperazone, meropenem, ofloxacin, cephalexin, cefepime HCl, cefuroxime sodium, minocycline HCl, cefaclor, cefazolin, trimethoprim and sulfamethoxazole, norfloxacin, trovafloxacin, cefpodoxime proxetil, cefdinir, cefixime, panipenem, ceftibuten, levofloxacin, cefoxopran HCl, amikacin sulfate, aztreonam, minocycline HCl, ticarcillin disodium or mixtures thereof.

Another preferred active ingredient can be selected from the class antibiotics including beta-lactamse inhibitor, quinolone, macrolide, cephalosporin, glycopeptide, cephem, immunoglobin tetracycline, fluoroquinolone aminoglycoside, and monobactam antibiotics.

Many of the active ingredients listed above have unpalatable tastes. Taste-masking of compositions with those unpalatable active materials is well-known in the art. The active ingredient may coated with a suitable polymer/fats/waxes/emulsifiers/carbohydrates. The use of flavors and sweeteners to mask the unpalatability of the active materials is also well-known. Thus, other materials which can be incorporated into composition include flavors, colors and sweeteners. Importantly, it is possible to incorporate high levels of flavors, sweeteners and other taste-masking agents, making the compositions more palatable when undesirable tastes accompany the active materials.

Taste masking may be chosen from natural and synthetic flavor liquids. Flavors useful include, without limitation, volatile oils, synthetic flavor oils, flavoring aromatics, oils, liquids, oleoresins or extracts derived from plants, leaves, flowers, fruits, stems and combinations thereof. A non-limiting list of examples include citrus oils such as lemon, orange, grape, lime and grapefruit and fruit essences including apple, pear, peach, grape, strawberry, raspberry, cherry, plum, pineapple, apricot or other fruit flavors.

Taste masking of the active ingredients can be done using the well-known processes in the art such as fluidization, spray drying, spray congealing, complex co-acervation, resin complexation, matrix granulation using carbohydrates, resins, polymers, waxes & fats.

Taste enhancers may be chosen from natural and synthetic flavor liquids. Flavors useful include, without limitation, volatile oils, synthetic flavor oils, flavoring aromatics, oils, liquids, oleoresins or extracts derived from plants, leaves, flowers, fruits, stems and combinations thereof. A non-limiting list of examples include citrus oils such as lemon, orange, grape, lime and grapefruit and fruit essences including apple, pear, peach, grape, strawberry, raspberry, cherry, plum, pineapple, apricot or other fruit flavors. fat

Other useful flavorings include aldehydes and esters such as benzaldehyde (cherry, almond), citral, i.e., alphacitral (lemon, lime), neral, i.e., betal-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), and mixtures thereof.

The compositions are “storage stable”, meaning that the compositions are stable in the absence of special handling procedures. The inventive compositions are stable both prior to packaging and after packaging. Importantly, the inventive compositions maintain their stability and integrity without refrigeration and without humidity controls being implemented during handling, packaging and storing of the products. Additionally, since the compositions exhibit increased integrity and stability, the compositions can be used in most of the current economical packages suitable for a global environment. Further, high temperatures are not needed when processing the packaging and sealing.

Without further description, it is believed that one of ordinary skill in the art can, using the preceding description and the following illustrative examples, make and use the present invention. The following examples are given to illustrate the present invention. It should be understood that the invention is not to be limited to the specific conditions or details described in the examples.

### Example 1

### General Procedure

The following procedure describes filling of two or more separate ingredients being filled into a single stick pack using the innovation claimed in this innovation.

The equipment consisted of a two-hopper system with two dosing wheels, vertical forming jaws, horizontal sealing jaws, stick forming tube, and heaters.

The dosing wheels were made of fixed thickness to accommodate different fill weights. Volume of the dosing cup determined the fill weight of each ingredient into the stick (final package). The accuracy of fill was achieved by measuring the density of the product being dosed and the required dosage. Volume of the cup was determined to achieve the required fill weight. Based on the size of the granules/beads and the thickness of the dosing disc, the diameter of the dosing hole was determined. The dosing wheels were placed on a central vertical shaft and had holes made on the periphery. The size of the hole determined the maximum fill weight that can be achieved with the fixed depth (X) wheel. The components were filled on to the respective dosing wheels through the hopper. The wheels rotated at a set speed filling the components into their cups. The scrapers took off extra components on top of the cup and ensure complete accurate fill of the ingredient. The dosing wheels moved further and emptied their contents into the funnel located below the dosing wheels. The components were thus dosed into the stick (final package). The film rolled to form the stick sealed on the bottom and sides. After filling of the required components, the horizontal sealing jaws pulled the stick downward and sealed and cut the stick from the top, while forming the second stick to repeat the process.

Two hoppers, with two dosing wheels, were used for two dose operation to form a dosage form containing guaifenesin encapsulated beads and rapid melt granules. The components were dosed into the packet to form a unit dose.

### Example 2

### Placebo Rapid Melt Granules

Placebo (not containing an active ingredient) rapid melt granules (can also be powders, beads, minitablets, pellets, nanoparticles, or combinations thereof) can be made using the following formulations:

Placebo Rapid Melt Granules Formulation 1

| Ingredients | Quantity by weight (%) |
|---|---|
| Sugar 6x | 40.26 |
| Bakers Special Sugar | 35.00 |
| Sucralose Micronized | 0.50 |
| Natural Seal Orange flavor | 2.54 |
| Polyplasdone XL-10 | 3.00 |
| Cocoa Butter | 12.00 |
| S-Maz 60K (Monoemul-60) | 3.00 |
| Carbowax Sentry PEG 3350, | 3.00 |
| Granular | |
| Stepanol WA 100 | 0.05 |
| Tween 80K | 0.15 |
| Syloid 244FP | 0.50 |
| Total | 100.00% |

Placebo Rapid Melt Granules Formulation 2

| Ingredients | Quantity by weight (%) |
|---|---|
| Diluents | |
| Mannitol Powder Pearlitol 160C | 39.90 |
| Sorbitol Powder Neosorb P60W | 35.00 |
| Emcocel 90M | 15.00 |
| Sweetening Agent | |
| Sucralose Powder | 0.50 |
| Flavor | |
| Bubble Gum FL S/D Powder # | 2.50 |
| 209V47 | |
| Disintegrants | |
| Polyplasdone XL-10 | 4.00 |
| Sodium Starch Glycolate | 2.00 |
| Emulsifiers | |
| Tween 80K | 0.25 |
| Sodium Lauryl Sulfate | 0.10 |
| Binder | |
| Plasdone K 29/32 | 0.75 |
| Purified USP water | |
| Total | 100.00% |

Placebo Rapid Melt Granules Formula 3

| Ingredients | Quantity by weight (%) |
|---|---|
| Diluents | |
| Mannitol Powder Pearlitol 160C | 81.15 |
| Emcocel 90M | 10.00 |
| Sweetening Agent | |
| Sucralose Powder | 0.50 |
| Flavor | |
| Bubble Gum FL S/D Powder # | 2.50 |
| 209V47 | |
| Disintegrants | |
| Polyplasdone XL-10 | 5.00 |
| Emulsifiers | |
| Tween 80K | 0.15 |
| Sodium Lauryl Sulfate | 0.10 |
| Binder | |
| Plasdone K 29/32 | 0.60 |
| Purified USP water | |
| Total | 100.00% |

The above formulations can be made as granules, powders, beads, minitablets, pellets, nanoparticles, or combinations thereof for use in the packs of the present invention. Although the flavors are specified in the formulations, it can be changed as desired without adversely compromising the fast melting property of the formulation. The formulations of this example can be used in the subsequent examples.

### Example 3

### Guaifenesin/Rapid Melt Granules in a Delivery Form

The general procedure of example 1 is carried out for weighing 180 mg of encapsulated guaifenesin beads and 780 mg of placebo rapid melt granules—grape flavor.

Further, the general procedure of Example 1 was carried out for packaging and sealing to give a fill weight 960 mg per dosage form for guaifenesin rapid melt granules—grape flavor. The flavored rapid melt granules dissolved in the mouth within less than 5 seconds and the saliva took away the encapsulated guaifenesin beads. Thus, the rapid melt granules helped to swallow the encapsulated beads.

Component 1: Beads of encapsulated Guaifenesin having bulk density 0.70 g/mL, Particle size: retained above #16 is 0.1%, above #20 is 30.2%, above #25 is 65.4%, above #35 is 4.2%.

Component 2: flavored Placebo rapid melt granules with bulk density 0.6 g/mL and particle size between #8 mesh and #60 mesh.

Assay is performed on the packaged products to verify the dose accuracy, assay is 99%. Also, the compositions are stable both prior to packaging and after packaging.

The Weight Variation is checked on the packaged product:

Weight variation for Placebo Guaifenesin 50 mg Rapid melt
granules - Grape flavor (960 mg) Packaged

| Sachets S. | | Avg. | Relative standard |
|---|---|---|---|
| No | Weight (mg) | Weight (mg) | deviation (RSD) |
| 1 | 958.9 | 953.9 | 0.8 |
| 2 | 953.2 | | |
| 3 | 955.1 | Min | Max |
| 4 | 952 | 942.1 | 971.5 |
| 5 | 957.5 | | |
| 6 | 950.9 | Target | |
| 7 | 953.1 | 960 | mg |
| 8 | 958.1 | Batch# | |
| 9 | 953.2 | CPI-198PL-001 and CPI-181C-005 | |
| 10 | 959.9 | Acceptance criteria | |
| 11 | 944.9 | | |
| 12 | 946.5 | | |
| 13 | 960.9 | | |
| 14 | 956.4 | Low limit | High limit |
| 15 | 955.2 | 888 | 1032 |
| 16 | 949 | Below 722 mg | Above 839 mg |
| 17 | 943.1 | 0 | 0 |
| 18 | 950.8 | Allowable limit | ±7.5% |
| 19 | 971.5 | | |
| 20 | 945.4 | Actual variation | <±2.0% |
| 21 | 949.2 | | |
| 22 | 970.4 | | |
| 23 | 966.8 | | |
| 24 | 942.1 | | |
| 25 | 942.9 | | |

Weight variation for Guaifenesin 50 mg Rapid melt
granules (180 mg) Packaged

| Sachets | | | |
|---|---|---|---|
| S. No | Wt (mg) | Avg Wt (mg) | RSD |
| 1 | 180.5 | 181.3 | 1.3 |
| 2 | 178.1 | | |
| 3 | 182.4 | Min | Max |
| 4 | 179.1 | 177.8 | 187.2 |
| 5 | 180.1 | | |

| | | |
|---|---|---|
| 6 | 181.2 | Target |

| | | | |
|---|---|---|---|
| 7 | 180.8 | 180 | mg |

| | | |
|---|---|---|
| 8 | 184 | Batch# |
| 9 | 183.9 | CPI-181C-005 |
| 10 | 183.5 | Acceptance criteria |
| 11 | 180.7 | |
| 12 | 182.9 | |
| 13 | 179 | |

| | | | |
|---|---|---|---|
| 14 | 181.6 | Low limit | High limit |
| 15 | 180.9 | 171 | 189 |
| 16 | 182.7 | Below 171 mg | Above 189 mg |
| 17 | 187.2 | 0 | 0 |
| 18 | 179.4 | Allowable | ±5% |
| 19 | 179 | limit | |
| 20 | 178.8 | | |
| 21 | 185.2 | | |
| 22 | 177.8 | | |
| 23 | 181.8 | | |
| 24 | 179.4 | | |
| 25 | 183.2 | | |

Weight variation for Placebo Guaifenesin 50 mg
Rapid melt granules - Grape flavor (780 mg)
Packaged

| Sachets | | | |
|---|---|---|---|
| S. No | Wt (mg) | Avg Wt (mg) | RSD |
| 1 | 793.1 | 783.7 | 1.0 |
| 2 | 780.6 | | |
| 3 | 788.9 | Min | Max |
| 4 | 789.6 | 765.9 | 796.5 |
| 5 | 781.1 | | |

| | | |
|---|---|---|
| 6 | 780.2 | Target |

| | | | |
|---|---|---|---|
| 7 | 788.6 | 780 | mg |

| | | |
|---|---|---|
| 8 | 771.8 | Batch# |
| 9 | 767 | CPI-198PL-001 |
| 10 | 765.9 | Acceptance criteria |
| 11 | 783.6 | |
| 12 | 794.5 | |
| 13 | 779.4 | |

| | | | |
|---|---|---|---|
| 14 | 779.7 | Low limit | High limit |
| 15 | 783.1 | 722 | 839 |
| 16 | 785.3 | Below 722 mg | Above 839 mg |
| 17 | 785.8 | 0 | 0 |
| 18 | 793.7 | Allowable | ±7.5% |
| 19 | 783.9 | limit | |
| 20 | 796.5 | Actual | <±2.2 |
| 21 | 777.2 | Variation | |
| 22 | 782.8 | | |
| 23 | 793 | | |
| 24 | 784.8 | | |
| 25 | 782.6 | | |

Weight variation for Guaifenesin 50 mg Rapid melt
granules (180 mg) Packaged

| Sachets | | | |
|---|---|---|---|
| S. No | Wt (mg) | Avg Wt (mg) | RSD |
| 1 | 185.4 | 179.8 | 1.8 |
| 2 | 182.8 | | |
| 3 | 177.4 | Min | Max |
| 4 | 185.5 | 172.5 | 185.5 |
| 5 | 180.6 | | |

| | | |
|---|---|---|
| 6 | 180 | Target |

| | | | |
|---|---|---|---|
| 7 | 182.3 | 180 | mg |

| | | |
|---|---|---|
| 8 | 182.1 | Batch# |
| 9 | 179.3 | CPI-181C-004 |
| 10 | 177.5 | Acceptance criteria |
| 11 | 178.7 | |
| 12 | 177.7 | |
| 13 | 175.5 | |

| | | | |
|---|---|---|---|
| 14 | 181 | Low limit | High limit |
| 15 | 176 | 171 | 189 |
| 16 | 180.9 | Below 171 mg | Above 189 mg |
| 17 | 172.5 | 0 | 0 |
| 18 | 178.9 | Allowable | ±5% |
| 19 | 177.3 | limit | |
| 20 | 182.1 | | |
| 21 | 179.6 | | |
| 22 | 177.1 | | |
| 23 | 178.6 | | |
| 24 | 180.7 | | |
| 25 | 185.4 | | |

The amount of guaifenesin per dosage form can be 50 mg or 100 mg and fill weight of encapsulated guaifenesin beads 30% was changed accordingly. The fill weight for flavored rapid melt granules can be 300 mg to 1500 mg.

The compositions in packet are stable over the months.

### Example 4

### Diphenhydramine HCl 25 mg/Rapid Melt Granules in a Delivery Form

The general procedure of Example 1 was carried out for weighing 250 mg of diphenhydramine HCl beads 10% and 550 mg placebo rapid melt granules—black cherry flavor. Further, the general procedure of Example 1 was carried out for packaging and sealing to give fill weight 800 mg per dosage form for diphenhydramine HCl 25 mg rapid melt granules—black cherry flavor.

Component 1: Diphenhydramine HCl beads 10%
Bulk density: 0.75 g/ml
Particle size: % retained on #40 is 3.9%, #60 is 13.0%, #80 is 21.8% and #100 is 9.6%
Component 2: Flavored Placebo Rapid melt granules:
Bulk density: 0.6 g/mL
Particle size: NLT 75% retained on #35 mesh

### Example 5

### Acetaminophen 80 mg/Rapid Melt Granules in a Delivery Form

The general procedure of Example 1 was carried out for weighing 88 mg of Encapsulated Acetaminophen beads 91% and 812 mg of placebo rapid melt granules—grape flavor. Further, the general procedure of Example 1 was carried out for packaging and sealing to give fill weight 900 mg per dosage form of acetaminophen 80 mg rapid melt granules—grape flavor.

### Component 1: Encapsulated Acetaminophen 91%

Bulk density: 0.49 g/ml
Particle size: % retained on #40 is 28% and on #80 is 70%
Component 2: Flavored Placebo Rapid melt granules:
Bulk density: 0.6 g/mL
Particle size: NLT 75% retained on #35 mesh

### Example 6

### Acetaminophen 80 mg/Rapid Melt Granules in a Delivery Form

The general procedure of Example 1 was carried out for weighing, packaging and sealing for 1100 mg of acetaminophen granules or beads (rapid melt granules) to give fill weight 1100 mg per dosage form for acetaminophen 80 mg rapid melt granules.

### Example 7

### Acetaminophen 160 mg/Rapid Melt Granules in a Delivery Form

The general procedure of Example 1 was carried out for weighing 176 mg of Encapsulated Acetaminophen beads 91% and 812 mg of placebo rapid melt granules—grape flavor. Further, the general procedure of Example 1 was carried out for packaging and sealing to give fill weight of 988 mg per dosage form of acetaminophen 160 mg rapid melt granules—grape flavor.

### Component 1: Encapsulated Acetaminophen 91%

Bulk density: 0.49 g/ml
Particle size: % retained on #40 is 28% and on #80 is 70%
Component 2: Flavored Placebo Rapid melt granules:
Bulk density: 0.6 g/mL
Particle size: NLT 75% retained on #35 mesh

### Example 8

### Acetaminophen 160 mg/Rapid Melt Granules in a Delivery Form

The general procedure of Example 1 was carried out for weighing, packaging and sealing for 1212 mg of acetaminophen granules or beads (rapid melt granules)—grape flavor to give fill weight of 1212 mg per dosage form of acetaminophen 160 mg rapid melt granules—grape flavor.

### Example 9

### Acetaminophen 325 mg/Rapid Melt Granules in a Delivery Form

The general procedure of Example 1 was carried out for weighing 357 mg of Encapsulated Acetaminophen beads 91% and 812 mg of placebo rapid melt granules—grape flavor. Further, the general procedure of example 1 is carried out for packaging and sealing to give fill weight 1169 mg per dosage form for acetaminophen 325 mg rapid melt granules—grape flavor.

### Component 1: Encapsulated Acetaminophen 91%

Bulk density: 0.49 g/ml
Particle size: % retained on #40 is 28% and on #80 is 70%
Component 2: Flavored Placebo Rapid melt granules:
Bulk density: 0.6 g/mL
Particle size: NLT 75% retained on #35 mesh

### Example 10

### Acetaminophen 325 mg/Rapid Melt Granules in a Delivery Form

The general procedure of Example 1 was carried out for weighing, packaging and sealing for 1457.5 mg of acetaminophen granules or beads (rapid melt granules)—grape flavor to give fill weight 1457.5 mg per dosage form for acetaminophen 325 mg rapid melt granules.

### Example 11

### Acetaminophen 500 mg/Rapid Melt Granules in a Delivery Form

The general procedure of Example 1 was carried out for weighing 549 mg of Encapsulated Acetaminophen beads 91% and 1001 mg of placebo rapid melt granules—grape flavor. Further, the general procedure of Example 1 was carried out for packaging and sealing to give fill weight 1550 mg per dosage form for acetaminophen 500 mg rapid melt granules—grape flavor.

### Component 1: Encapsulated Acetaminophen 91%

Bulk density: 0.49 g/ml
Particle size: % retained on #40 is 28% and on #80 is 70%
Component 2: Flavored Placebo Rapid melt granules:
Bulk density: 0.6 g/mL
Particle size: NLT 75% retained on #35 mesh

### Example 12

### Acetaminophen 500 mg/Rapid Melt Granules in a Delivery Form

The general procedure of Example 1 was carried out for weighing, packaging and sealing for 1900 mg of acetaminophen granules or rapid melt granules—grape flavor to give 1900 mg fill weight per dosage form for acetaminophen 500 mg rapid melt granules.

### Example 13

### Acetaminophen 650 mg/Rapid Melt Granules in a Delivery Form

The general procedure of Example 1 was carried out for weighing 714 mg of Encapsulated Acetaminophen beads 91% and 1001 mg of placebo rapid melt granules—grape flavor. Further, the general procedure of Example 1 was carried out for packaging and sealing to give 1714 mg fill weight per dosage form for acetaminophen 650 mg rapid melt granules—grape flavor.

### Component 1: Encapsulated Acetaminophen 91%

Bulk density: 0.49 g/ml
Particle size: % retained on #40 is 28% and on #80 is 70%
Component 2: Flavored Placebo Rapid melt granules:
Bulk density: 0.6 g/mL
Particle size: NLT 75% retained on #35 mesh

### Example 14

### Acetaminophen 650 mg/Rapid Melt Granules in a Delivery Form

The general procedure of Example 1 was carried out for weighing, packaging and sealing for 2000 mg of acetaminophen granules or beads (rapid melt granules)—grape flavor in dosage form to give fill weight 2000 mg per dosage form for acetaminophen 650 mg rapid melt granules.

### Example 15

### Benzocaine 6 mg/Rapid Melt Granules—Grape Flavor in a Delivery Form

The general procedure of Example 1 was carried out for weighing 500 mg of benzocaine 6 mg rapid melt granules—grape flavor, for packaging and sealing to give benzoaine 6 mg grape flavor in a dosage form.

Bulk density of benzocaine rapid melt granules: 0.7 g/mL
Particle size: % retained on #16 is 6.1%, % retained on #25 is 90.6%, % retained on #35 is 3.0%, % retained on #40 is 0.1% and passed through #40 is 0.3%
After packaging the analytical testing is conducted to verify the dose accuracy.
Assay is 100.8% which shows the dose accuracy.

### Example 16

### Extended Release Mesalamine in a Delivery Form

The general procedure of Example 1 was carried out for weighing 1773 mg of mesalamine extended release beads, for packaging and sealing to give extended release mesalamine 500 mg beads in a dosage form.

Particle size: passed through #16 mesh and retained on #25 mesh
Bulk density: 0.8 g/mL

The amount of mesalamine per dosage form can be, from about 200 mg to about 2000 mg, including specific intermediate amounts such as 250 mg, 300 mg, 400 mg, 500 mg, 600 mg, 750 mg, 1000 mg, 1200 mg, 1500 mg, and 1800 mg. The fill weight of mesalamine extended release beads per dosage form can be 500 mg to 5000 mg. The flavored placebo rapid melt granules can optionally be used with mesalamine extended release beads.

### Example 17

### Extended Release Metoprolol Succinate in a Delivery Form

The general procedure of Example 1 was carried out for weighing 700 mg metoprolol succinate extended release beads, for packaging and sealing to give extended release metoprolol succinate 200 mg beads in a delivery form.

Particle size: passed through #25 mesh and retained on #40 mesh
Bulk density: 0.55-0.65 g/mL

The amount of metoprolol succinate per dosage form can be 25 mg, 50 mg, 100 mg or 200 mg. The fill weight of metoprolol succinate extended release beads per dosage form will change accordingly. The flavored placebo rapid melt granules can optionally be used with metoprolol succinate extended release beads.

### Example 18

### Extended Release Diltiazem in a Delivery Form

The general procedure of Example 1 was carried out for weighing 1285 mg of diltiazem extended release beads, for packaging and for sealing to give extended release diltiazem 420 mg beads in a delivery form.

Particle size: Passed through #14 mesh and retained on #20 mesh
Bulk Density: 0.65 g/mL-0.75 g/mL

The amount of diltiazem per dosage form can be 120 mg, 180 mg, 240 mg, 300 mg, 360 mg or 420 mg. The fill weight of diltiazem extended release beads per dosage form can be 200 mg to 1500 mg. The flavored placebo rapid melt granules can optionally be used with diltiazem extended release beads.

### Example 19

### Extended Release Balsalazide 2.2 g in a Delivery Form

The general procedure of Example 1 was carried out for weighing 2.8 g of balsalazide extended release beads, for packaging and for sealing to give extended release balsalazid 2.2 g dosage form in a delivery form.

The balsalazide 2.2 g can optionally be filled in delivery form with flavored placebo rapid melt granules 2.8 g in a dosage form such as packet or a sealed container.

### Example 20

### Amoxicillin and Clavulanate Potassium in a Delivery Form

The general procedure of Example 1 was carried out for weighing taste masked amoxicillin (152 g) and clavulanate potassium (32 g). Further, the general procedure of Example 1 was carried out for packaging and for sealing to give amoxicillin 125 mg and clavulanate potassium 32 mg beads in a dosage form in a sealed container.

The amount of Amoxicillin and Clavulanate Potassium per dosage form can be 125/32 mg, 200/29 mg, 250/63 mg, and 400/57 mg. The fill weight of Amoxicillin and Clavulanate Potassium beads per dosage form in a sealed container can be 400 mg to 1500 mg. The flavored placebo rapid melt granules can optionally be used with amoxicillin and clavulanate potassium.

### Example 21

### Validation for Guaifenesin 50 mg and Grape Flavor Rapid Melt Granules

The general procedure of Example 1 was carried out for packaging and sealing to give a fill weight of 922 mg, which included a blend of encapsulate guaifenesin coated beads and rapid melt granules (grape flavor). Each packet contained 178 mg of encapsulated guaifenesin coated beads (equivalent to 50 mg of guaifenesin). The flavored rapid melt granules dissolved in the mouth within less than 5 seconds; and the saliva took away the encapsulated guaifenesin beads.

Component 1: Encapsulated Guaifenesin Beads (CPI-181C) were spherical to near spherical shaped, white to off-white colored free flowing beads of sizes varying between 0.80-1.30 mm.

Component 2: Rapid melt granules—Grape Flavor was white to off white colored free flowing granules.

Three batches were performed and the results were as follows (weight variations and content uniformity were tested in accordance with USP32-NF 27 <905>):

| | | | | |
|---|---|---|---|---|
| Tests | Specifications | Batch 1 | Batch 2 | Batch 3 |

Content Uniformity (CU) Sample Testing

| | | | | |
|---|---|---|---|---|
| Weight Variation | 922 mg ± 7.5% (853 mg to 991 mg) | Min: 937 mg | Min: 942 mg | Min: 942 mg |
| | | Max: 968 mg | Max: 962 mg | Max: 961 mg |
| Content | Acceptance Value NMT 15.0 | CU1: 13.4 | CU1: 4.2 | CU1: 3.0 |
| Uniformity | | CU2: 9.1 | CU2: 4.0 | CU2: 5.1 |
| | | CU3: 5.4 | CU3: 13.6 | CU3: 4.4 |
| | | CU4: 4.7 | CU4: 10.1 | CU4: 8.4 |

Composite Sample Testing

| Description | Each Packet contains white to off-white | | Conforms | Conforms | Conforms |
|---|---|---|---|---|---|
| | colored, spherical to near-spherical free | | | | |
| | flowing beads. | | | | |
| Identification | A. | Evaporated filtrate of Guaifenesin | Conforms | Conforms | Conforms |
| | | and chloroform upon treatment | | | |
| | | with few drops of Formaldehyde | | | |
| | | and Sulfuric Acid gives a deep | | | |
| | | Cherry-Red to Purple color. | | | |
| | B. | Retention time of the | Conforms | Conforms | Conforms |
| | | Guaifenesin peak in the | | | |
| | | Chromatogram of the Assay | | | |
| | | preparation corresponds to that | | | |
| | | of the Guaifenesin Peak in the | | | |
| | | chromatogram of the standard | | | |
| | | preparation. | | | |

| | | | | |
|---|---|---|---|---|
| Assay | Each Packet contains Guaifenesin NLT | 54 mg | 51 mg | 50 mg |
| | 45 mg to NMT 55 mg | | | |
| Average Weight | Each Packet contains 922 mg ± 5% | 978 mg | 962 mg | 962 mg |
| | (876 mg to 986 mg) | | | |
| Weight Variation | 922 mg ± 7.5% (853 mg to 991 mg) | Ave: 958 mg | Ave: 956 mg | Ave: 957 mg |
| | | Min: 949 mg | Min: 943 mg | Min: 952 mg |
| | | Max: 963 mg | Max: 962 mg | Max: 962 mg |
| Content | Acceptance Value NMT 15.0 | 8.4 | 5.9 | 4.5 |
| Uniformity | | | | |
| Related | β isomer: NMT 1.5% | 0.4% | 0.4% | 0.4% |
| Substances | Guaiacol: NMT 0.03% | 0.00% | 0.00% | 0.00% |
| | Other Impurities: NMT 0.5% | 0.1% | 0.1% | 0.1% |
| | Total Impurities: NMT 1.0% | 0.1% | 0.1% | 0.1% |
| Microbiological | Total Bacteria Count: Less than 1000 cfu/g | Less than 10 cfu/g | Less than 10 cfu/g | Less than 10 cfu/g |
| Testing | Yeast and Mold: Less than 1000 cfu/g | Less than 10 cfu/g | Less than 10 cfu/g | Less than 10 cfu/g |
| | E. coli: Negative in 10 g | Negative in 10 g | Negative in 10 g | Negative in 10 g |
| | Salmonella: Negative in 10 g | Negative in 10 g | Negative in 10 g | Negative in 10 g |
| | Staph. Aureus: Negative in 10 g | Negative in 10 g | Negative in 10 g | Negative in 10 g |
| | P. aeruginosa: Negative in 10 g | Negative in 10 g | Negative in 10 g | Negative in 10 g |

The following table showed the yield for the three batches:

| | | Process Waste | | % Process Yield | |
|---|---|---|---|---|---|
| | | | Rapid melt | | Rapid melt |
| Batch # | Theoretical Yield | Guaifenesin | granules | Guaifenesin | granules |
| 1 | 39,325 Sticks | 4507 sticks | 1650 sticks | 85%* | 93% |
| 2 | 39,325 Sticks | 565 Sticks | 565 Sticks | 95% | 93% |
| 3 | 39,325 Sticks | 1055 Sticks | 1055 Sticks | 96% | 93% |

*yield of encapsulated guaifenesin beads in validation batch #1 was low due to accidental spillage.

### Example 22

### Validation for Guaifenesin 100 mg and Bubble Gum Flavor Rapid Melt Granules

The general procedure of Example 1 was carried out for packaging and sealing to give a fill weight of 1100 mg, which included a blend of encapsulate guaifenesin coated beads and rapid melt granules (bubble gum flavor). Each packet contained 356 mg of encapsulated guaifenesin coated beads (equivalent to 100 mg of guaifenesin). The flavored rapid melt granules dissolved in the mouth within less than 5 seconds; and the saliva took away the encapsulated guaifenesin beads.

Component 1: Encapsulated Guaifenesin Beads (CPI-181C) were spherical to near spherical shaped, white to off-white colored free flowing beads of sizes varying between 0.80-1.30 mm.

Component 2: Rapid melt granules (bubble gum flavor) was white to off white colored free flowing granules.

Six batches were performed and the results were as follows (weight variations and content uniformity were tested in accordance with USP32-NF 27 <905>):

Batches 1-3

| | | | | |
|---|---|---|---|---|
| Tests | Specifications | Batch #1 | Batch #2 | Batch #3 |

Content Uniformity (CU) Sample Testing

| | | | | |
|---|---|---|---|---|
| Weight Variation | 1100 mg ± 7.5% (1018 mg to 1183 mg) | CU1: | CU1: | CU1: |
| | | Min: 1099 mg | Min: 1066 mg | Min: 1104 mg |
| | | Max: 1127 mg | Max: 1166 mg | Max: 1109 mg |
| | | CU2: | CU2: | CU2: |
| | | Min: 1051 mg | Min: 1078 mg | Min: 1103 mg |
| | | Max: 1099 mg | Max: 1113 mg | Max: 1110 mg |
| | | CU3: | CU3: | CU3: |
| | | Min: 1066 mg | Min: 1072 mg | Min: 1106 mg |
| | | Max: 1084 mg | Max: 1107 mg | Max: 1115 mg |
| | | CU4: | CU4: | CU4: |
| | | Min: 1064 mg | Min: 1068 mg | Min: 1104 mg |
| | | Max: 1107 mg | Max: 1102 mg | Max: 1112 mg |
| Content | Acceptance Value NMT 15.0 | CU1: 7.9 | CU1: 4.5 | CU1: 5.6 |
| Uniformity | | CU2: 9.0 | CU2: 9.6 | CU2: 3.3 |
| | | CU3: 7.9 | CU3: 6.1 | CU3: 5.6 |
| | | CU4: 11.6 | CU4: 9.3 | CU4: 7.8 |

Composite Sample Testing

| | | | | |
|---|---|---|---|---|
| Description | Each Packet contains white to off-white | Conforms | Conforms | Conforms |
| | colored, spherical to near-spherical free | | | |
| | flowing beads. | | | |
| Identification | A. Evaporated filtrate of Guaifenesin | Conforms | Conforms | Conforms |
| | and chloroform upon treatment | | | |
| | with few drops of Formaldehyde | | | |
| | and Sulfuric Acid gives a deep | | | |
| | Cherry-Red to Purple color. | | | |
| | C. Retention time of the | Conforms | Conforms | Conforms |
| | Guaifenesin peak in the | | | |
| | Chromatogram of the Assay | | | |
| | preparation corresponds to that | | | |
| | of the Guaifenesin Peak in the | | | |
| | chromatogram of the standard | | | |
| | preparation. | | | |
| Assay | Each Packet contains Guaifenesin NLT | 103 mg | 100 mg | 101 mg |
| | 90 mg to NMT 110 mg | | | |
| Average Weight | Each Packet contains 1100 mg ± 5% | 1070 mg | 1081 mg | 1133 mg |
| | (1045 mg to 1155 mg) | | | |
| Weight Variation | 1100 mg ± 7.5% (1018 mg to 1183 mg) | Min: 1059 mg | Min: 1062 mg | Min: 1100 mg |
| | | Max: 1079 mg | Max: 1098 mg | Max: 1123 mg |
| Content | Acceptance Value NMT 15.0 | 9.2 | 11.2 | 4.0 |
| Uniformity | | | | |
| Related | β isomer: NMT 1.5% | 0.4% | 0.4% | 0.4% |
| Substances | Guaiacol: NMT 0.03% | 0.00% | 0.00% | 0.00% |
| | Other Impurities: NMT 0.5% | 0.2% | 0.2% | 0.2% |
| | Total Impurities: NMT 1.0% | 0.4% | 0.4% | 0.3% |
| Water Activity | NMT 0.75 Aw | 0.39 Aw | 0.56 Aw | 0.58 Aw |
| Microbiological | Total Bacteria Count: Less than 1000 cfu/g | Less than 10 cfu/g | Less than 10 cfu/g | Less than 10 cfu/g |
| Testing | Yeast and Mold: Less than 1000 cfu/g | Less than 10 cfu/g | Less than 10 cfu/g | Less than 10 cfu/g |
| | E. coli: Negative in 10 g | Negative in 10 g | Negative in 10 g | Negative in 10 g |
| | Salmonella: Negative in 10 g | Negative in 10 g | Negative in 10 g | Negative in 10 g |
| | Staph. Aureus: Negative in 10 g | Negative in 10 g | Negative in 10 g | Negative in 10 g |
| | P. aeruginosa: Negative in 10 g | Negative in 10 g | Negative in 10 g | Negative in 10 g |

Batches 4-6

| | | | | |
|---|---|---|---|---|
| Tests | Specifications | Batch #4 | Batch #5 | Batch #6 |

Content Uniformity Sample Testing

| | | | | |
|---|---|---|---|---|
| Weight Variation | 1100 mg ± 7.5% (1018 mg to 1183 mg) | CU1: | CU1: | CU1: |
| | | Min: 1102 mg | Min: 1117 mg | Min: 1125 mg |
| | | Max: 1129 mg | Max: 1142 mg | Max: 1141 mg |
| | | CU2: | CU2: | CU2: |
| | | Min: 1115 mg | Min: 1122 mg | Min: 1107 mg |
| | | Max: 1131 mg | Max: 1134 mg | Max: 1138 mg |
| | | CU3: | CU3: | CU3: |
| | | Min: 1107 mg | Min: 1111 mg | Min: 1130 mg |
| | | Max: 1134 mg | Max: 1134 mg | Max: 1153 mg |
| | | CU4: | CU4: | CU4: |
| | | Min: 1103 mg | Min: 1097 mg | Min: 1122 mg |
| | | Max: 1123 mg | Max: 1116 mg | Max: 1143 mg |
| Visual Inspection | Vertical and Horizontal Seals shall be | CU1: Good | CU1: Good | CU1: Good |
| (3 sticks) | aligned uniformly and offer resistance | CU2: Good | CU2: Good | CU2: Good |
| | while trying to open it. | CU3: Good | CU3: Good | CU3: Good |
| | | CU4: Good | CU4: Good | CU4: Good |
| Leak Test | All stick shall pass leak test | CU1: Passed | CU1: Passed | CU1: Passed |
| | | CU2: Passed | CU2: Passed | CU2: Passed |
| | | CU3: Passed | CU3: Passed | CU3: Passed |
| | | CU4: Passed | CU4: Passed | CU4: Passed |

Composite Sample Testing

| | | | | |
|---|---|---|---|---|
| Description | Each Packet contains white to off-white | Conforms | Conforms | Conforms |
| | colored, spherical to near-spherical free | | | |
| | flowing beads. | | | |
| Identification | A. Evaporated filtrate of Guaifenesin | Conforms | Conforms | Conforms |
| | and chloroform upon treatment | | | |
| | with few drops of Formaldehyde | | | |
| | and Sulfuric Acid gives a deep | | | |
| | Cherry-Red to Purple color. | | | |
| | D. Retention time of the | Conforms | Conforms | Conforms |
| | Guaifenesin peak in the | | | |
| | Chromatogram of the Assay | | | |
| | preparation corresponds to that | | | |
| | of the Guaifenesin Peak in the | | | |
| | chromatogram of the standard | | | |
| | preparation. | | | |
| Assay | Each Packet contains Guaifenesin NLT | 103 mg | 101 mg | 101 mg |
| | 90 mg to NMT 110 mg | | | |
| Average Weight | Each Packet contains 1100 mg ± 5% | 1117 mg | 1113 mg | 1133 mg |
| | (1045 mg to 1155 mg) | | | |
| Weight Variation | 1100 mg ± 7.5% (1018 mg to 1183 mg) | Min: 1105 mg | Min: 1112 mg | Min: 1116 mg |
| | | Max: 1117 mg | Max: 1123 mg | Max: 1123 mg |
| Content | Acceptance Value NMT 15.0 | 4.4 | 6.0 | 5.2 |
| Uniformity | | | | |
| Related | β isomer: NMT 1.5% | 0.4% | 0.4% | 0.4% |
| Substances | Guaiacol: NMT 0.03% | 0.00% | 0.00% | 0.00% |
| | Other Impurities: NMT 0.5% | 0.2% | 0.2% | 0.2% |
| | Total Impurities: NMT 1.0% | 0.3% | 0.4% | 0.3% |
| Water Activity | NMT 0.75 Aw | 0.34 Aw | 0.25 Aw | 0.32 Aw |
| Microbiological | Total Bacteria Count: Less than 1000 cfu/g | Less than 10 cfu/g | NA | NA |
| Testing (To be | Yeast and Mold: Less than 1000 cfu/g | Less than 10 cfu/g | | |
| done only if water | E. coli: Negative in 10 g | Negative in 10 g | | |
| activity fails) | Salmonella: Negative in 10 g | Negative in 10 g | | |
| | Staph. Aureus: Negative in 10 g | Negative in 10 g | | |
| | P. aeruginosa: Negative in 10 g | Negative in 10 g | | |

The following table showed the yield for the six batches:

| | | Process Waste | | % Process Yield | |
|---|---|---|---|---|---|
| | | | Rapid melt | | Rapid melt |
| Batch # | Theoretical Yield | Guaifenesin | granules | Guaifenesin | granules |
| 1 | 19,662 Sticks | 209 sticks | 496 sticks | 99% | 100% |
| 2 | 19,662 Sticks | 916 sticks | 920 sticks | 95% | 95% |
| 3 | 19,662 Sticks | 212 sticks | 588 sticks | 99% | 100% |
| 4 | 19,662 Sticks | 228 sticks | 739 sticks | 99% | 96% |
| 5 | 19,662 Sticks | 62 sticks | 958 sticks | 100% | 95% |
| 6 | 19,662 Sticks | 695 sticks | 1767 sticks | 96% | 91% |

### Example 23

### Guaifenesin 100 mg and Dextromethorphan HBr 5 mg/Rapid Melt Granules in a Delivery Form

The general procedure of Example 1 was carried out for packaging and sealing to give a fill weight of 1300 mg, which included a blend of encapsulate coated beads containing guaifenesin and dextromethorphan Hbr and placebo rapid melt granules (orange flavor). Each packet contained 356 mg of encapsulated guaifenesin and dextromethorphan HBr coated beads (equivalent to 100 mg of guaifenesin & 5 mg of dextromethorphan HBr). The flavored rapid melt granules dissolved in the mouth within less than 5 seconds; and the saliva took away the encapsulated guaifenesin and dextromethorphan HBr beads.

Component 1: Encapsulated guaifenesin and dextromethorphan HBr beads were semi-spherical to near oblong shaped, white to off-white colored free flowing beads of sizes varying between 0.595-1.19 mm. Bulk density is 0.5-0.7 g/mL.

Component 2: Rapid melt granules (orange flavor) was white to off white colored free flowing granules. Bulk density: 0.6 g/mL. Particle size: NLT 75% retained on #35 mesh

The guaifenesin and dextromethorphan HBr coated beads were made by first making guaifenesin and dextromethorphan HBr uncoated beads using the following formulation:

Guaifenesin and dextromethorphan
HBr uncoated beads

| | Quantity |
|---|---|
| Ingredients | (weight %) |
| Guaifenesin USP | 30.00 |
| Dextromethorphan HBr USP | 1.50 |
| Emcocel 90M (Microcrystalline cellulose) | 18.80 |
| Emcocel 90M (Microcrystalline cellulose) | 18.80 |
| Lactose Anhydrous, NF DT | 20.00 |
| (Lactose) | |
| Starch 1500 | 8.00 |
| (Pregelatinised starch) | |
| Plasdone K-29/32 | 2.00 |
| (Povidone) | |
| Stepanol WA-100 | 0.40 |
| (Sodium Lauryl Sulfate) | |
| Tween 80K (Polysorbate 80) | 0.30 |
| Syloid 244 FP | 0.20 |
| (Silicondioxide) | |
| Purified water | + |
| TOTAL | 100.00% |

+ indicates the component evaporated in the final product

The uncoated guaifenesin and dextromethorphan HBr is then coated using the following formulation:

Guaifenesin and dextromethorphan
HBr coated beads

| | Quantity |
|---|---|
| Ingredients | (weight %) |
| Guaifenesin 30% Dextromethorphen 1.5% | 93.88 |
| Uncoated beads | |
| Eudragit L100 55 | 3.70 |
| Triethyl Citrate NF | 0.37 |
| Talc | 1.85 |
| Sylloid 244FP | 0.20 |
| Isopropyl Alcohol, USP* | + |
| TOTAL | 100.00% |

### Example 24

### Diphenhydramine HCL 12.5 mg/Rapid Melt Granules in a Delivery Form

The general procedure of Example 1 was carried out for packaging and sealing to give a fill weight of 900 mg, which included a blend of encapsulated diphenhydramine HCl granules 16% and rapid melt granules (cherry flavor). Each packet contained 78.27 mg of encapsulated diphenhydramine HCl granules (equivalent to 12.5 mg of Diphenhydramine HCl). The flavored rapid melt granules dissolved in the mouth within less than 5 seconds; and the saliva took away the encapsulated diphenhydramine HCl granules.

Component 1: Encapsulated diphenhydramine HCl Granules were spherical to near spherical, white to off-white colored free flowing granules of sizes varying between 0.125-0.595 mm. Bulk density is 0.5-0.7 g/mL.

Component 2: Rapid melt granules (cherry flavor) was white to off white colored free flowing granules. Bulk density: 0.6 g/mL. Particle size: NLT 75% retained on #35 mesh.

The diphenhydramine HCl coated granules were made by first making the uncoated granules using the following formulation:

Uncoated diphenhydramine HCl

| | Quantity |
|---|---|
| | (weight |
| Ingredients | %) |
| Diphenhydramine HCl | 20.00 |
| USP | |
| Emcocel 90M | 62.00 |
| (Microcrystalline cellulose | |
| USP) | |
| Emcompress (Dibasic, | 12.00 |
| Calcium Phosphate) | |
| Surelease E-7-19040 | 4.00 |
| (Ethylcellulose Aqueous | |
| Dispersion) | |
| Splenda Sucralose | 1.00 |
| Syloid 244 FP (Silicon | 1.00 |
| dioxide) | |
| Purified Water USP | + |
| TOTAL | 100.00 |

The uncoated diphenhydramine HCl granules are then double coated using the following formulation to produce the encapsulated diphenhydramine HCl:

Encapsulated diphenhydramine HCl

| | Quantity |
|---|---|
| | (weight |
| Ingredients | %) |
| Diphenhydramine HCl uncoated | 79.85 |
| Granules 20% | |

Coating I

| | |
|---|---|
| Surelease E-7-19040^{†} | 10.00 |
| Triacetin USP | 2.00 |
| Talc, USP | 2.00 |
| Purified water* | * |
| Purified water* | * |

Coating II

| | |
|---|---|
| Eudragit L30D-55^{‡} | 3.50 |
| Triacetin USP | 0.70 |
| Talc, USP | 1.75 |
| Purified water* | * |
| Purified water* | * |
| Syloid 244FP | 0.20 |
| Total | 100.00 |

### Example 25

### Diphenhydramine HCL 25 mg/Rapid Melt Granules in a Delivery Form

The general procedure of Example 1 was carried out for packaging and sealing to give a fill weight of 1100 mg, which included a blend of encapsulated diphenhydramine HCl granules 16% and rapid melt granules (cherry flavor). Each packet contained 156.54 mg of encapsulated diphenhydramine HCl granules (equivalent to 25 mg of diphenhydramine HCl). The flavored rapid melt granules dissolved in the mouth within less than 5 seconds; and the saliva took away the encapsulated diphenhydramine HCl granules.

Component 1: Encapsulated Diphenhydramine HCl Granules were spherical to near spherical, white to off-white colored free flowing granules of sizes varying between 0.125-0.595 mm. Bulk density is 0.5-0.7 g/mL.

Component 2: Rapid melt granules (Cherry flavor) was white to off white colored free flowing granules. Bulk density: 0.6 g/mL. Particle size: NLT 75% retained on #35 mesh.

### Example 26

### Diphenhydramine HCL 50 mg/Rapid Melt Granules in a Delivery Form

The general procedure of Example 1 was carried out for packaging and sealing to give a fill weight of 1300 mg, which included a blend of encapsulated diphenhydramine HCl granules 16% and rapid melt granules (cherry flavor). Each packet contained 313.08 of encapsulated diphenhydramine HCl granules (equivalent to 50 mg of Diphenhydramine HCl). The flavored rapid melt granules dissolved in the mouth within less than 5 seconds; and the saliva took away the encapsulated diphenhydramine HCl granules.

Component 1: Encapsulated Diphenhydramine HCl Granules were spherical to near spherical, white to off-white colored free flowing granules of sizes varying between 0.125-0.595 mm. Bulk density is 0.5-0.7 g/mL.

Component 2: Rapid melt granules (Cherry flavor) was white to off white colored free flowing granules. Bulk density: 0.6 g/mL. Particle size: NLT 75% retained on #35 mesh.

### Example 27

### Ibuprophen 100 mg/Rapid Melt Granules in a Delivery Form

The general procedure of Example 1 was carried out for packaging and sealing to give a fill weight of 1100 mg, which included a blend of encapsulated ibuprofen crystals 87% and rapid melt granules (orange flavor). Each packet contained 115 mg of encapsulated ibuprofen crystals (equivalent to 100 mg of ibuprofen). The flavored rapid melt granules dissolved in the mouth within less than 5 seconds; and the saliva took away the encapsulated ibuprofen crystals.

Component 1: Encapsulated ibuprofen crystals were semi-spherical to near oblong shaped, white to off-white colored free flowing granules. Bulk density is 0.5-0.7 g/mL.

Component 2: Rapid melt granules (Orange flavor) was white to off white colored free flowing granules. Bulk density: 0.6 g/mL. Particle size: NLT 75% retained on #35 mesh.

The ibuprofen crystals were encapsulated using the following formulation:

Encapsulated ibuprofen crystals

| | Ind. |
|---|---|
| | Qty. |
| | (% |
| Material Name | w/w) |
| Ibuprofen Crystals | 88.90 |
| Primary coating Coating | |
| Polymers | |
| Eudragit L 100 55 | 8.00 |
| Plasticizer | |
| Triethyl Citrate | 1.50 |
| Tachy agent | |
| Purified Talc | 1.00 |
| Coating Solvent | |
| Isopropyl Alcohol | + |
| (Commertial) ml (6% solution | |
| wb of Eudragit/v) | |
| Sylloid 244 FP | 0.60 |
| Total | 100.00 |
| Second Coating | |
| Coating Polymers | |
| Part 1Coated Ibuprofen | 96.00 |
| (88.9%) | |
| Eudragit S100 | 3.00 |
| Plastisizer | |
| Triethyl citrate | 0.20 |
| Tachy agent | |
| Purified Talc | 1.00 |
| Coating Solvent | |
| Isopropyl Alcohol | + |
| (commertial) | |
| Total | 100.00 |

### Example 28

### Ibuprophen 100 mg/Rapid Melt Granules in a Delivery Form

The general procedure of Example 1 was carried out for packaging and sealing to give a fill weight of 1300 mg, which included a blend of encapsulated ibuprofen crystals 87% and rapid melt granules (orange flavor). Each packet contained 230 mg of encapsulated ibuprofen crystals (equivalent to 100 mg of Ibuprofen). The flavored rapid melt granules dissolved in the mouth within less than 5 seconds; and the saliva took away the encapsulated ibuprofen crystals.

Component 1: Encapsulated Ibuprofen Crystals were semi-spherical to near oblong shaped, white to off-white colored free flowing granules. Bulk density is 0.5-0.7 g/mL.

Component 2: Rapid melt granules (Orange flavor) was white to off white colored free flowing granules. Bulk density: 0.6 g/mL. Particle size: NLT 75% retained on #35 mesh.

### Example 29

### Ibuprophen 100 mg/Rapid Melt Granules in a Delivery Form

The general procedure of Example 1 was carried out for packaging and sealing to give a fill weight of 1300 mg, which included a blend of encapsulated ibuprofen beads 28.5% and rapid melt granules (grape flavor). Each packet contained 356 mg of encapsulated ibuprofen beads (equivalent to 100 mg of ibuprofen). The flavored rapid melt granules dissolved in the mouth within less than 5 seconds; and the saliva took away the encapsulated ibuprofen beads.

Component 1: Encapsulated ibuprofen beads were spherical to oblong shaped, white to off-white colored free flowing beads. Bulk density is 0.5-0.7 g/mL.

Component 2: Rapid melt granules (Orange flavor) was white to off white colored free flowing granules. Bulk density: 0.6 g/mL. Particle size: NLT 75% retained on #35 mesh.

The encapsulated ibuprofen beads were made by first making the uncoated beads using the following formulation:

Ibuprofen uncoated beads

| | Quantity |
|---|---|
| Ingredients | (weight %) |
| Ibuprofen | 31.50 |
| Emcocel 90M (Microcrystalline | 18.80 |
| cellulose) | |
| Emcocel 90M (Microcrystalline | 18.80 |
| cellulose) | |
| Lactose Anhydrous, NF DT | 20.00 |
| (Lactose) | |
| Starch 1500 | 8.00 |
| (Pregelatinised starch) | |
| Plasdone K-29/32 | 2.00 |
| (Povidone) | |
| Stepanol WA-100 | 0.40 |
| (Sodium Lauryl Sulfate) | |
| Tween 80K (Polysorbate 80) | 0.30 |
| Syloid 244 FP | 0.20 |
| (Silicondioxide) | |
| Purified water | + |
| TOTAL | 100.00% |

The uncoated ibuprofen beads are then coated using the following formulation to produce the encapsulated ibuprofen beads:

Ibuprofen coated beads

| | Quantity |
|---|---|
| Ingredients | (weight %) |
| Ibuprofen Uncoated beads | 93.88 |
| Eudragit L100 55 | 3.70 |
| Triethyl Citrate NF | 0.37 |
| Talc | 1.85 |
| Sylloid 244FP | 0.20 |
| Isopropyl Alcohol, USP* | + |
| TOTAL | 100.00% |

### Example 30

### Dextromethorphan Hbr 7.5 mg/Rapid Melt Granules in a Delivery Form

The general procedure of Example 1 was carried out for packaging and sealing to give a fill weight of 800 mg, which included a blend of encapsulated dextromethorphan HBr powder 31.83% and rapid melt granules (cherry flavor). Each packet contained 23.56 mg of encapsulated dextromethorphan HBr powder (equivalent to 7.5 mg of dextromethorphan HBr). The flavored rapid melt granules dissolved in the mouth within less than 5 seconds; and the saliva took away the encapsulated dextromethorphan HBr powder.

Component 1: Encapsulated dextromethorphan HBr powder was light brown to brown color powder.

Component 2: Rapid melt granules (Cherry flavor) was white to off white colored free flowing granules. Bulk density: 0.6 g/mL. Particle size: NLT 75% retained on #35 mesh.

The encapsulated dextromethorphan HBr powder was made using the following formulation:

Encapsulated Dextromethorphan HBr
Powder

| | Qty. |
|---|---|
| Material Name | (w/w %) |
| Adsorbant/Resin | |
| Amberlite IRP88** | 70.00 |
| Active | |
| Dextromethorphan HBr | 30.00 |
| Solvent | |
| Purified water* | |
| Total | 100.00 |

### Example 31

### Dextromethorphan Hbr 15 mg/Rapid Melt Granules in a Delivery Form

The general procedure of Example 1 was carried out for packaging and sealing to give a fill weight of 1000 mg, which included a blend of encapsulated dextromethorphan HBr powder 31.83% and rapid melt granules (cherry flavor). Each packet contained 47.13 mg of encapsulated dextromethorphan HBr powder (equivalent to 15 mg of dextromethorphan Hbr). The flavored rapid melt granules dissolved in the mouth within less than 5 seconds; and the saliva took away the encapsulated dextromethorphan HBr powder.

Component 1: Encapsulated Dextromethorphan Hbr powder was light brown to brown color powder.

Component 2: Rapid melt granules (cherry flavor) was white to off white colored free flowing granules. Bulk density: 0.6 g/mL. Particle size: NLT 75% retained on #35 mesh.

### Example 32

### Dextromethorphan Hbr 30 mg/Rapid Melt Granules in a Delivery Form

The general procedure of Example 1 was carried out for packaging and sealing to give a fill weight of 1300 mg, which included a blend of encapsulated dextromethorphan HBr powder 31.83% and rapid melt granules (cherry flavor). Each packet contained 94.25 mg of encapsulated dextromethorphan HBr powder (equivalent to 30 mg of dextromethorphan HBr). The flavored rapid melt granules dissolved in the mouth within less than 5 seconds; and the saliva took away the encapsulated dextromethorphan HBr powder.

Component 1: Encapsulated Dextromethorphan HBr powder was light brown to brown color powder.

Component 2: Rapid melt granules (Cherry flavor) was white to off white colored free flowing granules. Bulk density: 0.6 g/mL. Particle size: NLT 75% retained on #35 mesh.

### Example 33

### Dextromethorphan HBr 30 mg/Rapid Melt Granules in a Delivery Form

The general procedure of Example 1 was carried out for packaging and sealing to give a fill weight of 1300 mg, which included a blend of encapsulated dextromethorphan HBr beads and rapid melt granules (cherry flavor). Each packet contained 356 mg of encapsulated dextromethorphan HBr beads (equivalent to 30 mg of dextromethorphan HBr). The flavored rapid melt granules dissolved in the mouth within less than 5 seconds; and the saliva took away the encapsulated dextromethorphan HBr beads.

Component 1: Encapsulated dextromethorphan HBr beads were white to off white spherical to near spherical shaped beads. Bulk density is 0.5-0.7 g/mL.

Component 2: Rapid melt granules (cherry flavor) was white to off white colored free flowing granules. Bulk density: 0.6 g/mL. Particle size: NLT 75% retained on #35 mesh.

Encapsulated dextromethorphan HBr beads were made by first making uncoated beads using the formulation below:

Dextromethorphan HBr Uncoated Beads

| | Quantity |
|---|---|
| Ingredients | (weight %) |
| Dextromethorphan HBr USP | 30.00 |
| Emcocel 90M (Microcrystalline cellulose) | 19.55 |
| Emcocel 90M (Microcrystalline cellulose) | 19.55 |
| Lactose Anhydrous, NF DT | 20.00 |
| (Lactose) | |
| Starch 1500 | 8.00 |
| (Pregelatinised starch) | |
| Plasdone K-29/32 | 2.00 |
| (Povidone) | |
| Stepanol WA-100 | 0.40 |
| (Sodium Lauryl Sulfate) | |
| Tween 80K (Polysorbate 80) | 0.30 |
| Syloid 244 FP | 0.20 |
| (Silicondioxide) | |
| Purified water | + |
| TOTAL | 100.00% |

The dextromethorphan HBr uncoated beads were then coated using the formulation below to produce encapsulated dextromethorphan HBr beads:

Encapsulated Dextromethorphan HBr Beads

| | Quantity |
|---|---|
| Ingredients | (weight %) |
| Dextromethorphan Hbr Uncoated beads | 93.88 |
| Eudragit L100 55 | 3.70 |
| Triethyl Citrate NF | 0.37 |
| Talc | 1.85 |
| Sylloid 244FP | 0.20 |
| Isopropyl Alcohol, USP* | + |
| TOTAL | 100.00% |

### Example 34

### Caffeine 50 mg/Rapid Melt Granules in a Delivery Form

The general procedure of Example 1 was carried out for packaging and sealing to give a fill weight of 1100 mg, which included a blend of encapsulated caffeine 40% and rapid melt granules (cherry flavor). Each packet contained 125 mg of encapsulated encapsulate caffeine (equivalent to 50 mg of caffeine). The flavored rapid melt granules dissolved in the mouth within less than 5 seconds; and the saliva took away the encapsulated caffeine powder.

Component 1: Encapsulated caffeine was light yellow to yellow colored free flowing powder.

Component 2: Rapid melt granules (cherry flavor) was white to off white colored free flowing granules. Bulk density: 0.6 g/mL. Particle size: NLT 75% retained on #35 mesh.

The encapsulated caffeine was made using the formulation below:

Encapsulated Caffeine

| | Qty. |
|---|---|
| Material Name | (weight %) |
| Part-I | |
| Wax | |
| Carnuba Wax | 55.00 |
| Active Ingredient | |
| Caffeine Anhydrous | 40.00 |
| USP | |
| Plasticizer | |
| Dist. Acetylated | 5.00 |
| Monoglyceride 5-07 | |
| (Myvacet 5-07) | |
| Total | 100.00 |

### Example 35

### Caffeine 100 mg/Rapid Melt Granules in a Delivery Form

The general procedure of Example 1 was carried out for packaging and sealing to give a fill weight of 1500 mg, which included a blend of encapsulated caffeine 40% and rapid melt granules (cherry flavor). Each packet contained 250 mg of encapsulated caffeine powder (equivalent to 100 mg of caffeine). The flavored rapid melt granules dissolved in the mouth within less than 5 seconds; and the saliva took away the encapsulated caffeine.

Component 1: Encapsulated Caffeine was light yellow to yellow colored free flowing powder.

Component 2: Rapid melt granules (Cherry flavor) was white to off white colored free flowing granules. Bulk density: 0.6 g/mL. Particle size: NLT 75% retained on #35 mesh.

### Example 36

### Phenylephrine HCL 5 mg/Rapid Melt Granules in a Delivery Form

The general procedure of Example 1 was carried out for packaging and sealing to give a fill weight of 750 mg, which included a blend of encapsulated phenylephrine HCl granules 10% and rapid melt granules (orange flavor). Each packet contained 50 mg of encapsulated phenylephrine HCl granules (equivalent to 5 mg of phenylephrine HCl). The flavored rapid melt granules dissolved in the mouth within less than 5 seconds; and the saliva took away the encapsulated phenylephrine HCl granules.

Component 1: Encapsulated phenylephrine HCl granules were spherical to near spherical, white to off-white colored free flowing granules.

Component 2: Rapid melt granules (Orange flavor) was white to off white colored free flowing granules. Bulk density: 0.6 g/mL. Particle size: NLT 75% retained on #35 mesh.

The encapsulated phenylephrine HCl granules were made in two steps. First, the phenylephrine HCl is granulated using the formulation in Part A below. Second, the granulated phenylephrine HCl granules were then encapsulated using the formulation shown in Part B.

Encapsulated Phenylephrine HCl Granules

| Ingredient Name | Qty. (weight %) |
|---|---|
| Part A - Drug resin granulation | |
| Phenylephrine HCl | 31.66 |
| Amberlite IRP88 (Polacrilin | 63.33 |
| Potassium, Methacrylic acid & | |
| Divinylbenzene polymer) | |
| Purified Water (*) | + |
| Part B - Ethyl Cellulose | |
| granulation | |
| Drug Resin Granules from Part A | (95.00) |
| Ethocel Standard 4 Premium | 5.00 |
| (Ethyl Cellulose 4 cps) | |
| Isopropyl Alcohol USP | + |
| Total | 100.00 |

### Example 37

### Phenylephrine HCL 10 mg/Rapid Melt Granules in a Delivery Form

The general procedure of Example 1 was carried out for packaging and sealing to give a fill weight of 900 mg, which included a blend of encapsulated phenylephrine HCl granules 10% and rapid melt granules (orange flavor). Each packet contained 100 mg of encapsulated phenylephrine HCl granules (equivalent to 10 mg of phenylephrine HCl). The flavored rapid melt granules dissolved in the mouth within less than 5 seconds; and the saliva took away the encapsulated phenylephrine HCl granules.

Component 1: Encapsulated Phenylephrine HCl Granules were spherical to near spherical, white to off-white colored free flowing granules.

Component 2: Rapid melt granules (orange flavor) was white to off white colored free flowing granules. Bulk density: 0.6 g/mL. Particle size: NLT 75% retained on #35 mesh.

### Example 38

### Acetaminophen 500 mg and Diphenhydramine HCl 25 mg/Rapid Melt Granules in a Delivery Form

The general procedure of Example 1 was carried out for packaging and sealing to give a fill weight of 1500 mg, which included a blend of encapsulate coated granules containing acetaminophen and diphenhydramine HCl granules and rapid melt granules (Grape flavor). Each packet contained 625 mg of encapsulated acetaminophen and diphenhydramine HCl granules (equivalent to 500 mg of acetaminophen and 25 mg of diphenhydramine HCl). The flavored rapid melt granules dissolved in the mouth within less than 5 seconds; and the saliva took away the encapsulated acetaminophen and diphenhydramine HCl granules.

Component 1: Encapsulated acetaminophen and diphenhydramine HCl granules were white to off-white colored free flowing granules.

Component 2: Rapid melt granules (grape flavor) was white to off white colored free flowing granules. Bulk density: 0.6 g/mL. Particle size: NLT 75% retained on #35 mesh.

Acetaminophen and diphenhydramine HCl granules were encapsulated using the formulation below:

Encapsulated Acetaminophen and
Diphenhydramine HCl Granules

| Material Name | % |
|---|---|
| Active drug | |
| Acetaminophen & | 92.60 |
| dDiphenhydramine HCl | |
| granules | |
| Polymer | |
| Methacrylic acid Co-polymer | 4.50 |
| (Eudragit L100-55) | |
| Plasticizer | |
| Triethylcitrate | 0.45 |
| Glident | |
| Purified Talc | 2.25 |
| Sylloid 244FP (Silicondioxide) | 0.20 |
| Solvent | |
| IPA USP (6% solid content) | + |
| IPA for homogenization | + |
| Total | 100.00 |

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A solid or semi-solid oral delivery dosage form comprising a first component and a second component contained in a sealed container.

2. The dosage form of claim 1, wherein the first component is an active ingredient and the second component is rapid melt granules.

3. The dosage form of claim 2, wherein the active ingredient is in the form of beads, minitablets, pellets, granules, crystals, powder, or combinations thereof.

4. The dosage form of claim 3, wherein the active ingredient is coated beads, mini tablets, granules, crystals, powder, or combinations there off.

5. The dosage form of claim 1, wherein the first component is an modified release or a taste masked active ingredient.

6. The dosage form of claim 1, wherein the sealed container is a packet, a sachet, a pouch, or a blister pack.

7. The dosage form of claim 1, wherein a batch to batch variation in the individual component is no more than 5%.

8. The dosage form of claim 1, further comprising a third component.

9. A method for making a solid or semi-solid dosage form comprising the steps of
(a) metering a volume of a first component;
(b) introduced the volume of the first component into a container;
(c) metering a volume of a second component;
(d) introducing the volume of the second component into the container; and
(e) sealing the container.

10. The method of claim 9, wherein the active ingredient is in the form of beads, minitablets, pellets, granules, crystals, powder, or combinations thereof.

11. The method of claim 9, wherein the active ingredient is in the form of coated beads, minitablets, pellets, granules, crystals, powder, or combinations thereof.

12. The method of claim 9, wherein the container is a packet, a sachet, a pouch, or a blister pack.

13. The method of claim 9, wherein step (a) involves filling cups on the periphery of a first dosing wheel from a first hopper containing the first component.

14. The method of claim 13, wherein step (b) involves rotating the first wheel into a position where the first component drops into a funnel located below the cups on the first wheel.

15. The method of claim 9, wherein step (c) involves filling cups on the periphery of a second wheel from a second hopper containing the second component.

16. The method of claim 15, wherein step (d) involves rotating the second wheel into a position where the second component drops into a funnel located below the cups on the second wheel.

17. The method of claim 15, wherein step (a) involves filling cups on an outer portion of a dosing wheel from a first hopper containing the first component.

18. The method of claim 17, wherein step (c) involves filling cups on an inner portion of the dosing wheel from a second hopper containing the first component.

19. A system for making the pharmaceutical dosage form comprising
a first dosing wheel having opened recesses on its peripheral;
first dosing cups being retained in the opened recesses, each first dosing cup containing a cylindrical disc having a through hole in its center;
a second dosing wheel having opened recesses on its peripheral;
second dosing cups being retained in the opened recesses, each second dosing cup containing a cylindrical disc having a through hole in its center; and
a funnel locating below the first and second dosing wheels;
wherein the first and second dosing wheels are mounted such that they are capable of emptying the contents of their respective dosing cup into a container.

20. The system of claim 19, wherein the first dosing wheel is associated with a first hopper for filling the cups of the first wheel.

21. The system of claim 20, wherein the first hopper contains an active ingredient.

22. The system of claim 19, wherein the second dosing wheel is associated with a second hopper for filling the cups of the second wheel.

23. The system of claim 22, wherein the second hopper contains rapid melt granules.

24. The system of claim 19, wherein the bottom of each opened recess contains a cover that is capable of being in a closed position to retain the content of the dosing cup or in an opened position to empty the content of the dosing cup.

25. The system of claim 19, wherein the container directs its contents to a package.

26. The system of claim 25, further comprising a sealer for sealing the package.

27. A system for making the pharmaceutical dosage form comprising
a dosing wheel having concentric outer and inner portions, each portion having opened recesses therein;
dosing cups being retained in the opened recesses, each first dosing cup containing a cylindrical disc having a through hole in its center;
a funnel locating below the second dosing wheels;
wherein the first and wheel is positioned such it is capable of emptying the contents of one inner dosing cup and one outer dosing cup into a container.

28. The system of claim 27, wherein each recess in the outer portion is paired with each recess in the inner portion such that they lay on a radius line of the dosing wheel.

29. The system of claim 27, wherein the outer portion is capable of receiving a component from a first hopper.

30. The system of claim 29, wherein the first hopper contains an active ingredient.

31. The system of claim 27, wherein the inner portion is capable of receiving a component from a second hopper.

32. The system of claim 31, wherein the second hopper contains rapid melt granules.

33. The system of claim 27, wherein the bottom of each opened recess contains a cover that is capable of being in a closed position to retain the content of the dosing cup or in an opened position to empty the content of the dosing cup.

34. The system of claim 27, wherein the container directs its contents to a package.

35. The system of claim 34, further comprising a sealer for sealing the package.
